(19) United States
(12) Patent Application Publication
(10) Pub. No.: US 2005/0142550 A1
(43) Pub. Date: Jun. 30, 2005

(54) Method of genetic screening using an amplifiable gene

(76) Inventors: Noelle-Anne Sunstrom, La Perouse (AU); Charles Bailey, Randwick (AU)

Correspondence Address: EDWARDS & ANGELL, LLP, P.O. BOX 55874, BOSTON, MA, 02205, US

(21) Appl. No.: 10/491,582

(22) PCT Filed: Oct. 3, 2002
(86) PCT No.: PCT/AU02/01352

(30) Foreign Application Priority Data
Oct. 3, 2001 (AU) PR8051

(51) Int. Cl. C12Q 1/68; C12P 19/34

(52) U.S. Cl. 435/6; 435/91.2

(57) ABSTRACT

The present invention pertains to genetic screening methods and related cells and genetic constructs. In particular, the invention relates to a method of screening cells for an alteration, typically an amplification, in the copy number of a nucleic acid of interest using an amplifiable nucleic acid linked to a reporter nucleic acid; a method of screening cells for increased expression of a polypeptide of interest derived from the nucleic acid of interest; and related cells and genetic constructs. The method allows for high throughput screening of recombinant cells expressing a polypeptide or protein of interest and, in particular, for screening of cells expressing the polypeptide or protein of interest at elevated levels.

### TECHNICAL FIELD

The present invention pertains to genetic screening methods and related cells and genetic constructs. In particular, the invention relates to a method of screening cells for an alteration, typically an amplification, in the copy number of a nucleic acid of interest using an amplifiable nucleic acid linked to a reporter nucleic acid; a method of screening cells for increased expression of a polypeptide of interest derived from the nucleic acid of interest; and related cells and genetic constructs. The method allows for high throughput screening of recombinant cells expressing a polypeptide or protein of interest and, in particular, for screening of cells expressing the polypeptide or protein of interest at elevated levels.

### BACKGROUND

Any discussion of the prior art throughout the specification should in no way be considered as an admission that such prior art is widely known or forms part of common general knowledge in the field.

To obtain a stable clone for recombinant protein production requires the transfection of cells with an expression vector encoding the desired gene of interest and a dominant genetic marker. Cells that have taken up the expression vector DNA survive in appropriate selection media. Typically, for the selection of stable transfectants, a selectable marker such as an antibiotic resistance gene is transfected along with the target gene of interest.

Many commonly used mammalian expression systems are based on stably transfected Chinese Hamster Ovary (CHO) cells and transfection efficiencies in this system range from 10-60% of cells taking up the vector DNA. However, a wide variation in recombinant gene expression exists among clones that stably incorporate the foreign DNA into the genome due to the position effect by which different regions of the chromosome modulate the expression of the transfected gene. A search for random high producers, however, is time-consuming and labour-intensive using conventional screening methods requiring immunodetection. Many hundreds, even thousands of tansfected clones are typically screened for random variation in recombinant protein production.

In addition to screening for high producing clones, an amplification strategy can be exploited to achieve a higher level of expression of a desired gene. This can be achieved by selecting for amplification of a co-expressed marker gene. Two widely used amplification systems employing CHO cells are the dihydrofolate reductase (DoF) (1) and glutamine synthetase (GS) (2) genes. Selective cycling in the presence of increasing concentrations of methotrexate (MTX), an inhibitor of DHFR function or methionine sulfoximine (MSX), an inhibitor of GS results in amplification of the integrated DNA and increased expression of the desired gene product.

The CHO-DHFR expression system requires the use of a mutant CHO cell line, lacking enzyme activity for dihydrofolate reductase (3) and must be grown in the presence of glycine, hypoxanthine and thymidine (GHT). The GS expression system is not effective in CHO cells due to the endogenous GS activity in these cells (4). In addition, there appears to be a limit to the amount of amplification that can be achieved which results in MTX and MSX resistant clones but no further increase in recombinant protein productivity (5,6).

We have investigated the use of the amplifiable gene, metallothionein (MT). Although CHO cells contain MT genes, they lack MT activity because the genes have become silenced as a result of DNA methylation (7) (8) (9). An expression vector harbouring the MT gene can be used for the co-amplification of a foreign gene (10). Strategies have been described for the selection and amplification of foreign gene expression in CHO cells employing a MT gene-containing expression vector (11,12). Further, the MT gene can also act as a reporter or marker gene due to the fact that it confers resistance to metals when expressed.

However, expression systems utilising the MT gene have the disadvantage that in some cells the endogenous MT gene is amplified in response to selection pressure i.e. addition of metal ion. The amplified endogenous MT gene is no longer silenced and the expression of this gene leads to “erroneous” selection of cells that have not been transformed with the exogenous MT construct (carrying the gene of interest). As a consequence, false positives are common and screening is labour intensive.

Reporter genes are well known. For example, the green fluorescent protein (GEP) has been used for monitoring gene expression and selection of cells expressing inducible gene products (13,14). Further, derivatives of GFP have been developed which fluoresce at different wavelengths and which can also be used as marker or reporter genes. Expression systems in which a reporter peptide is fused to a protein of interest are also well known. However, these systems have several disadvantages in that the peptide must be cleaved from the fusion protein and, further, it may interfere, for example, with the folding of the protein of interest or, if left attached to the protein, may inhibit binding of the protein to its substrate or ligand.

In light of the above, it is an object of the present invention to provide a selection method which will overcome or substantially ameliorate at least some of the deficiencies of the prior art or to provide a useful alternative.

### SUMMARY OF THE INVENTION

It has surprisingly been found that an amplifiable nucleic acid linked to a reporter nucleic acid can be used to screen cells having an altered (eg. amplified) copy number of a nucleic acid of interest. This system can also be used to rapidly screen cells for expression of a product of interest and in protocols for high throughput selection of cells producing high levels of a product of interest.

According to a first aspect, the present invention provides a method of identifying from a plurality of cells, a cell in which the copy number of a nucleic acid of interest is altered compared to others wherein
(a) the cell in which the copy number of the nucleic acid of interest is altered comprises the nucleic acid of interest and an amplifiable nucleic acid linked to a reporter nucleic acid; and
(b) the copy number of the nucleic acid of interest is correlated with the copy number of the amplifiable nucleic acid and the reporter nucleic acid; and
   wherein the method comprises the step of screening the plurality of cells for a cell having an alteration in copy number of the reporter nucleic acid and/or an alteration in expression of the product of the reporter nucleic acid compared to the other cells.

The skilled addressee will understand that the other cells in the plurality of cells may or may not possess the nucleic acid of interest, the amplifiable nucleic acid and the reporter nucleic acid.

The nucleic acid of interest and the amplifiable nucleic acid linked to the reporter nucleic acid may be on a single DNA molecule. In such a molecule, it is preferable that the nucleic acid of interest be 10,000 bp or less from the amplifiable nucleic acid. More preferably, the nucleic acid of interest is 1,000 bp or less from the amplifiable nucleic acid.

The skilled addressee will be aware of methods of identifying cells in which the copy number of a reporter nucleic acid is altered. These include, for example, screening candidate clones by Southern blot analysis or sequencing. However, it is well known in the field that an increase in the copy number of a nucleic acid is likely to be associated with an increase in expression of the product of the nucleic acid. As such, in accordance with the present invention, the preferred means of identifying a cell in which the copy number of the nucleic acid of interest has been altered is by screening for an alteration in expression of the product of the reporter nucleic acid.

Preferably, the alteration in expression of the product of the reporter nucleic acid correlates with the alteration in expression of the product of the nucleic acid of interest.

Typically, the alteration in copy number of the nucleic acid of interest is an amplification of the copy number. However, it will be clear to the skilled addressee that in certain circumstances one may wish to identify cells having a decrease in copy number. This might be achieved by adjusting, for example, a factor to which the amplifiable nucleic acid responds, eg. reducing the metal ion concentration in the cell's growth medium.

Preferably, the copy number of the amplifiable nucleic acid is regulated by the concentration of metal ion present in the growth medium of the cell. In such a system, since the copy number of the amplifiable nucleic acid is correlated with the copy number of the nucleic acid of interest, the copy number of the nucleic acid of interest will be controlled by the concentration of the metal ion. As indicated, in the present invention cells in which an alteration in copy number of the nucleic acid of interest has occurred will be identifiable by a change in the copy number of the reporter nucleic acid (or by a change in the level of the product of the reporter nucleic acid). More preferably, the copy number of the nucleic acid of interest is amplified by increasing the metal ion concentration in the growth medium and, most preferably, it is amplified in response to an increase in metal ion concentration from within a range of 1 μM to 100 μM. Most preferably, the increase in metal ion concentration is from 2.5 μM to 5.0 μM.

Preferably, the metal ion is cadmium, zinc, copper, cobalt or nickel ion and more preferably, it is cadmium or zinc ion.

Preferably, the amplifiable nucleic acid encodes a product which can act as a selectable marker.

Preferably the amplifiable nucleic acid encodes a metallothionein (MT), dihydrofolate reductase, glutamine synthetase gene, CAD, adenosine deaminase, adenylate deaminase, UMP synthetase, IMP 5′-dehydrogenase, zanthine-guanine phosphoribosyltransferase, mutant HGPRTase or mutant thymidine kinase, thymidylate synthetase, P-glycoprotein 170, ribonucleotide reductase, glutamine synthetase, asparagine synthetase, arginosuccinate synthetase, ornithine decarboxylase, HMG-CoA reductase, N-acetylglucosaminyl transferase, threonyl-tRNA synthetase, Na+, K+-ATPase. More preferably the amplifiable nucleic acid encodes the human MT gene and most preferably, the human MT gene is the human metallothionein IIA gene.

Preferably, the product of the reporter nucleic acid is detectable by flow cytometry, florescence plate reader, fluorometer, microscopy, the naked eye or phenotypic detection eg. ability to grow in the presence of an inhibitor such as neomycin, ampicillin, hygromycin, puromycin, bleomycin, zeocin or kanomycin. More preferably, it is detectable by flow cytometry using a florescence activated cell sorter (FACS).

Preferably, the reporter nucleic acid encodes a green fluorescent protein (GFP), or a derivative thereof such as, for example, an enhanced green fluorescent protein (GFP), a yellow fluorescent protein (YFP), an enhanced yellow fluorescent protein (EYFP), a blue fluorescent protein (BFP), an enhanced blue fluorescent protein (EBFP), a cyan fluorescent protein (CFP), an enhanced cyan fluorescent protein (ECFP) or a red fluorescent protein (dsRED). Most preferably, the fluorescent protein is EGFP. However, it will be clear to the skilled addressee that any fluorescent protein, and indeed any suitable reporter, can be used in the present invention.

The nucleic acid of interest and the amplifiable nucleic acid linked to the reporter nucleic acid may be inserted into the cell by any suitable method eg. transformation or transfection. The nucleic acids may, for example, be on a single DNA construct. Alternatively, for example, they may be on two constructs (one comprising the linked nucleic acids and the other comprising the nucleic acid of interest). Such constructs may be co-transformed or co-transfected into the cell. Without being bound by theory, it is proposed that the co-transformed/co-transfected constructs may recombine during transformation/transfection with the result that both are integrated at the same site in the cell's genomic DNA thus allowing the amplifiable nucleic acid to control the copy number of the nucleic acid of interest.

The skilled addressee will understand that any genetic construct(s) or system(s) may be used in the present invention provided the alteration in copy number of the nucleic acid of interest is correlated with the copy number of an amplifiable nucleic acid and a reporter nucleic acid wherein the amplifiable nucleic acid is linked to the reporter nucleic acid.

When the nucleic acids are on a single DNA construct, preferably the single DNA molecule is a plasmid. More preferably, it is a vector derived from the pNK plasmid. However, it will be understood that any suitable construct may be used.

Preferably the reporter nucleic acid is located downstream of the amplifiable nucleic acid. More preferably, the amplifiable nucleic acid is linked to the reporter gene by in frame fusion of the two nucleic acids such that a fusion product is produced. For example, in embodiments where MT is the amplifiable nucleic acid and GFP is the reporter nucleic acid, this type of linkage will result in the fusion product MRGFP.

However, it will be clear to the skilled addressee that any form of linkage which allows for the copy number of the reporter nucleic acid to be correlated with the copy number of the amplifiable nucleic acid and the nucleic acid of interest will function in the present invention. Further, the correlation between copy numbers of the nucleic acids utilised in the present invention need not be, and most often will not be, a 1:1 correlation. It is sufficient for the present invention that the copy number of the nucleic acids are correlated at least to some extent such that a change in copy number of one is reflected in a change in copy number of the other.

With this in mind, the nucleic acids may, for example, be linked by an internal ribosome entry site (IRES). An IRES allows for the production of a single transcript from two or more separate genes which can be translated into corresponding separate products due to the presence of an additional ribosome entry site(s) on the transcript. In a preferred embodiment, the IRES may be an attenuated IRES. An attentuated IRES is a derivative of IRES which results in a decrease in production of the product of the second gene with respect to the production of the product of the first. This has the advantage that, in a system in which the amplifiable nucleic acid is linked to the reporter nucleic acid by an attenuated IRES, for example, the product of the amplifiable nucleic acid could be produced in higher quantity than the product of the reporter nucleic acid. Hence, cells selected on the basis of an increase in the product of the reporter nucleic acid are likely to have high levels of expression of the amplifiable gene and, consequently, high levels of the nucleic acid of interest. In many cases, of course, high levels of the nucleic acid of interest will result in high levels of the product of the nucleic acid of interest.

Further, the reporter nucleic acid may be linked to the amplifiable nucleic acid by being cloned into an intron of the amplifiable nucleic acid.

It will be clear to the skilled addressee that one of the benefits of the present invention is that cells producing high levels of a product of the nucleic acid of interest (the product of interest) can be identified by reference to the product of the reporter nucleic acid and that, as such, the product of interest can be isolated without the need to cleave it from a selectable reporter peptide or protein. However, on the other hand, it is also to be understood that the invention is applicable to constructs in which the nucleic acid of interest is, itself linked to the amplifiable nucleic acid and the reporter nucleic acid for example, by in frame fusion. Depending on the requirements for the product of interest, the fusion product may include appropriate protease sites, or “tags” to aid purification, eg. 6-His tag or a glutathione-S-transferase tag or peptide epitopes that are readily detectable via specific antibodies such as the Flag and Hemophilus influenza epitopes.

The cell may be any cell type—prokaryotic or eukaryotic. Preferably, the cell is a mammalian cell. More preferably, it a suspension or attached Chinese Hamster Ovary cell (CHO). Most preferably, the cell is a CHOK1 cell.

The nucleic acid of interest may, of course, be any suitable nucleic acid including nucleic acid encoding, for example, an antibody, a biopharmaceutical, an endonuclease, a methylase, an oxidoreductase, a transferase, a hydrolase, a lysase, an isomerase or a ligase, a storage polypeptide, a transport protein, an antigen or antigenic determinant, a protective or defence protein, a hormone, a structural protein, a protease or a synthetic polypeptide of interest or part thereof.

According to a second aspect, the present invention provides a method of identifying a cell expressing a polypeptide of interest comprising:
(a) expressing the polypeptide of interest in a cell in which expression of the polypeptide is correlated with expression of a reporter nucleic wherein the reporter nucleic acid is linked to an amplifiable nucleic acid;
(b) identifying the cell expressing the polypeptide of interest from among other cells by monitoring expression of the reporter nucleic acid.

According to a third aspect, the present invention provides a method of isolating a polypeptide of interest comprising:
(a) transforming or transfecting cells with a construct or constructs harbouring the nucleic acid encoding the polypeptide of interest such that expression of the polypeptide of interest is correlated with expression of an amplifiable nucleic acid and a reporter nucleic acid and wherein the amplifiable nucleic acid and the reporter nucleic acid are linked;
(b) increasing expression of the amplifiable nucleic acid;
(c) selecting a cell expressing the product of the reporter nucleic acid; and
(d) isolating the polypeptide of interest therefrom.

Preferably the amplifiable nucleic acid is the gene encoding MT. Preferably the reporter nucleic acid is the gene encoding GFP or a derivative thereof. Most preferably, the MT and GFP genes are fused in frame.

It will be clear to the skilled addressee that this method can be used in robotic screening and in protocols for high throughput selection of cells producing high levels of a product of interest

Accordingly, in a fourth aspect, the present invention provides a cell comprising (a) a nucleic acid of interest, and (b) an amplifiable nucleic acid linked to a reporter nucleic acid, wherein the copy number of the nucleic acid of interest is correlated with the copy number of the amplifiable nucleic acid and the reporter nucleic acid.

According to a fifth aspect, the present invention provides a method of altering the copy number of a nucleic acid of interest in a cell according to the fourth aspect, comprising exposing the cell to a factor that alters the copy number of the amplifiable nucleic acid.

According to a sixth aspect, the present invention provides a construct comprising a nucleic acid of interest and an amplifiable nucleic acid linked to a reporter nucleic acid such that when the construct is present in a cell, the copy number of the nucleic acid of interest is correlated with the copy number of the amplifiable nucleic acid and the reporter nucleic acid.

According to a seventh aspect, the present invention provides a cell identified by a method according to the first or second aspect.

According to an eighth aspect, the present invention provides a polypeptide of interest isolated by a method according to the third aspect.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

### BRIEF DESCRIPTION OF THE FIGURES

FIG. 1. The pMTGFP mammalian expression vector. The fusion gene MTGFP is under the control of the wildtype metallothionein IIA promoter. A target gene of interest is cloned into the multiple cloning site downstream of the M2.6 metallothionein promoter and is followed by a sequence coding the SV40 polyadenylation site for downstream processing of mRNA. The gene encoding neomycin and kanamycin (Neo/Kan) confers resistance to G418 and kanamycin in mammalian and bacterial cells respectively under the control of their respective promoters {Bailey, Baig, et al. 1999 38 /id}.

FIG. 2. Mean relative fluorescence units (RFU) of pMTGFP-transfected pools of cells were plotted against metal concentrations used to select individual pools. Transfected CHOK1 cells were selected in metal and/or G418 selection. The mean RFU is the average fluorescence of 10,000 single, viable cells. Background fluorescence equivalent to that of non-transfected cells has been subtracted. Inset shows flow cytometry profiles of cells surviving metal and/or G418 selection. The mean fluorescence values for each pool were plotted as a function of metal concentration used for selection prior to analysis. 48 hours after transfection, cells were exposed to 100 μM zinc and increasing concentrations of cadmium as indicated on the right of each set in the absence and presence of G418 for 8 days. Symbols represent in the presence of G418 selection ∘, and in the absence of G418 selection, ●.

FIG. 3. Mean relative fluorescence units (RFU) and productivity of pMTGFP/CAT transfected pools of cells.

FIG. 3A. The mean RFU is the average fluorescence of 10,000 single, viable cells. Background fluorescence equivalent to non-transfected cells has been subtracted. RFUs for each pool were plotted against metal concentrations used to select individual pools. Symbols represent in the presence of G418 selection ∘, and in the absence of G418 selection, ●. Inset shows flow cytometry profiles of pMTGFP-transfected CHOK1 cells subject to metal and/or G418 selection as described in FIG. 2.

FIG. 3B. CAT expression from metal-selected pMTGFP-pools. CAT expression measured in pg/μg total protein was determined from pools selected with metal (stippled bars) and/or G418 (solid bars) as indicated on the abscissa Metal concentrations include 100 μM ZnSO₄ and 0, 1, 2, 4 and 6 μM CdCl₂.

FIG. 4A. Fluorescence profiles measured in flow cytometer for pools of (a) untransfected CHO cells, (b) CHO cells transfected with pMTGFP/hGH selected in media containing (b) G418, or (c) metal (100 μM ZnF⁺⁺+4 μM Cd⁺⁺). Gates 10¹, 10², 10³ and 10⁴ were set at corresponding levels of fluorescence and cells were sorted from within each gate into microtitre plates.

FIG. 4B. Fluorescence is linearly correlated to productivity. The specific productivity of 12 clones was plotted against relative fluorescence of each individual clone as measured in the fluorometer. Clones were sorted and grown in microtitre plates and the condition media were assayed for hGH using (correlation coefficient (R²)=0.79).

FIG. 5. A comparison of fluorescence using flow cytometer and fluorometer for CHO cells transfected with pNKGFP. Transfected pools were selected in metal consisting of 100 μM ZnSO₄ with CdCl₂ (1-6 μM) in the presence or absence of 400 μg/ml G418. Following selection, cells were harvested and either analysed for fluorescence in the flow cytometry or seeded into microtitre plates and analysed in the fluorometer.

FIG. 6. A high-throughput screening protocol for the rapid isolation of high-producing cell-lines.

### DESCRIPTION OF THE INVENTION

Many commonly used mammalian expression systems are based on stably transfected Chinese Hamster Ovary (CHO) cells. Stable clones producing high levels of recombinant protein are obtained after transfection of cells with an expression vector encoding the desired gene of interest and a dominant genetic marker. A search for random high producing clones may, however, be time-consuming and labour-intensive. In an effort to reduce the time and labour involved in the screening for high producers, we constructed a system in which an amplifiable nucleic acid is linked to a reporter nucleic acid and used for efficient screening of cells for an alteration in copy number of a nucleic acid of interest and/or an alteration in the level of production of a product encoded by the nucleic acid of interest.

In the system exemplified below, selection and amplification can be visually monitored, thus allowing efficient screening of recombinant gene-positive clones and resulting in selection of clones having a high level of expression of a product of interest following amplification. The representative amplifiable nucleic acid used was the human metallothionein (MT) gene and the reporter nucleic acid chosen was the green fluorescent protein (GFP) gene. The genes were linked by fusing the nucleic acids in frame to allow production of a fusion protein, MTGFP (referred to as “the fusion marker”).

The use of the fusion marker facilitates the screening of high-producing clones expressing a nucleic acid of interest by using flow cytometry. The method is demonstrated below by the expression of either human growth hormone (hGH) or chloramphenicol acetyl transferase (CAT).

The fluorescence-activated cell sorter (FACS) can easily screen a million cells and sort those cells producing higher levels of fluorescing protein—in the present example MTGFP. When amplification of the nucleic acid of interest is correlated with amplification of the MTGFP construct, those cells having amplified nucleic acid of interest can be identified by FACS screening relying on detection of fluorescence from the GOP comprised in the MTGFP fusion marker. Since the level of production of a product of interest is often a direct function of the copy number of the nucleic acid encoding the product of interest, selection of clones producing MTGFP is likely to provide clones producing the product of interest at high levels.

Cells transfected with the MTGFP construct respond to successive stepwise cadmium selection and amplification with increasing fluorescence that can be monitored using a flow cytometer or a fluorometer (a microtitre plate reader equipped with the appropriate filters to measure GFP fluorescence). Using clones isolated from increasing cadmium-resistant pools, we have demonstrated that the increase in fluorescence correlates linearly with increasing levels of recombinant product. An increase in fluorescence is evidence for an increase in productivity.

Clones in microtitre plates can be screened using the fluorometer without disturbing the integrity of the cultures and high-producers can be immediately identified. A high-throughput screening process using the MTGFP-encoding expression vector reduces dramatically the time and labour involved in screening large numbers of recombinant clones, especially if the procedure is adapted to robotic handling or automated procedures.

Expression of MTGFP gene acts as a dominant selectable marker allowing rapid and more efficient selection of clones at defined metal concentrations than the antibiotic G418. The MTGFP gene can be used as a selectable and amplifiable gene for the amplification of foreign gene expression.

Using the protocol described below it was possible to isolate cell lines reaching specific productivities of >30 μg/10⁶ cells /day within four weeks of selection/amplification in metal and fluorescence screening. In the past, the isolation of those few clones supporting such high levels of productivity were possible only after screening thousands of clones requiring years of person-hours. Such a screening method can be easily adapted to automated procedures using robotic handling systems, capable of managing tens of thousands of transfected clones in a fraction of time and effort that is required at present.

A preferred embodiment of the invention will now be described by way of example only, with reference to the accompanying figures.

### EXAMPLE 1

### Engineering the MTGFP Fusion Gene

The coding sequence for the enhanced Green Fluorescent Protein (eGFP, Clontech) was cloned in frame to the 3′ end of the human metallothionein IIA gene (MT)(15) using primer overlap extension PCR (16). For this purpose, four oligonucleotides were synthesized and are described as follows:

MTGFP-1:
5′ TAC TCT TCC TCC CTG CAG TCT CTA 3′;
MTGFP-2:
5′ CAC CAT GGG CCC GGC GCA GCA GCT GCA 3′;
MTGFP-3:
5′ GCC GGG CCC ATG GTG AGC AAG GGC GAG 3′;
MTGFP-4:
5′ ATT TAC GCC TGC AGA TAC AT 3′

MTGFP-1 anneals −758 to −735 nt of the gene encoding MT relative to the translation start and includes the PstI site (5′ CTGCA/G 3′).

MTGFP-2 anneals to +630 to +656 relative to the MT transcription start site. The stop codon TGA is replaced by the sequence 5′ CCCGGG 3′ encoding two additional amino acids proline and glycine as well as the recognition sequence for the restriction enzyme ApaI

MTGFP-3 anneals to −9 to +18 relative to the transcription start site of the coding sequence of eGFP. The ATG start codon is in frame with the MT gene sequence and is located within a 15 nucleotide tag homologous with MTGFP-2.

MTGFP4 anneals to +954 to +973 relative to the transcription start site of the coding sequence of eGFP, and includes the recognition site for the restriction enzyme PstI.

The eGFP gene was PCR amplified using MTGFP-3 and MTGFP-4 primers in a reaction mix containing Taq polymerase (Gibco-BRL), dNTPs (Progen), 2 mM Me²⁺ ions and 10% DMSO at an annealing temperature of 50° C. as described (16). Similarly, the MTIIA gene was amplified using the primers MTGFP-1 and MTGFP-2. The amplified products were gel purified and primer overlap extension was used to amplify the fusion MTGFP using primers MTGFP-1 and MTGFP-4.. The reaction required 2mM Mg²⁺ and 10% DMSO for the GC-rich Mt-encoding template DNA. The specificity was increased after 3 cycles by raising the annealing temperature from 50° C. to 55° C. The reaction yielded a 3281 base pair fragment of DNA.

### EXAMPLE 2

### Construction of Expression Vectors pMTGFP, pMTGFP/hGH and pMTGFP/CAT

The 2381 bp fragment containing the MTGFP coding sequence was digested with PstI to enable cloning into the expression vector pNK (12). After gel extraction the MTGFP containing fragment was cloned into pNKΔMT (MT gene deleted from pNK using PstI) to make pMTGFP. To construct pMTGFP/hGH, a 2223 bp EcoRI/KpnI fragment containing the genomic sequences of hGH (nt −559 to +2094 relative to the translational start site) was ligated to pMTGFP previously digested with the respective enzymes EcoRI and KpnI and transformed into DH5 bacteria. To construct pMTGFP/CAT, a 714 bp DNA fragment containing the coding region of CAT was obtained by digesting pNKCAT with HindIII and KpnI (12) and inserted into the respective sites in pMTGFP. DNA was isolated and purified from positive clones using anion exchange plasmid purification columns (Qiagen).

### EXAMPLE 3

### Cell Culture and Transfections

CHOK1 cells used to establish cell lines were derived from CHOK1 ATCC CCL61. All cells were grown in a complete medium (DMEM/Coons F12 mix (CSL) supplemented with 10% FCS (CSL). Cells were seeded into 35 mm plates 24 hours prior to transfection. For transactions, Lipofectamine 2000 (Life Technology) was used to transfect cells using optimum conditions for DNA and reagent mixes according to the manufacturer's protocol. Medium was removed 24 hours following transfection and replaced with fresh complete medium and plates were incubated for an additional 24 hours. The cells were then detached using EDTA/PBS and transferred to a T75 flask in fresh complete medium containing 400 μg/ml G418.

### EXAMPLE 4

### Metal Amplification

Cells from G418^{R} pools (surviving selection in 400 μg/ml G418 for X days) were grown in stepwise increasing amounts of metal (12) at an initial concentration 2.5 μM CdCl₂ and 50 μM ZnSO₄. The cells were passaged at 90% confluence 4 times before the concentration of CdCl₂ was doubled. Fresh ZnSO₄ was added to the medium at a concentration of 50 μM at all times. At each level of cadmium resistance the fluorescence of each pool was monitored using the flow cytometer and specific productivity was determined using ELISA.

### EXAMPLE 5

### Metal Selection

Following transfections of pMTGFP containing plasmids, CHO cells were passaged into 100 mm plates containing 7 mls of complete medium and were allowed to attach for six hours. Metal (1-10 μM CdCl₂ and 100 μM ZnSO₄) was added to the medium in the presence or absence of 400 μg/ml G418. The cells were monitored daily for emergent colonies of metal-resistant cells. Approximately 6 days after metal was added, the medium was removed and replaced with complete medium containing 100 μM ZnSO₄ and 2 μM CdCl₂. Once cultures reached confluence fluorescence was analysed using flow cytometry and recombinant protein levels were determined by ELISA.

### EXAMPLE 6

### Flow Cytometry FACS Sorting and Fluorometer

Flow cytometry was performed using a MoFlo Cytometer (Cytomation, Colo., USA) equipped with a multi-line argon laser emitting light at 488 nm. Analysis was performed using the CyCLOPS Summit operating system. When sorting was required Sortmaster software was used to determine correct drop delay and CyCLONE software that controlled a robotic arm to sort cells into microtitre plates. The flow cytometer was calibrated and optically aligned using Flow-Checks Fluorospheres (Beckman Coulter) before each analysis. Cells to be sorted were trypsinized and resuspended in complete medium and filtered through nylon mesh. Cells were analyzed at a flow rate of 1000 cell/s. Single, viable cells were determined using forward and side scatter. Typically, cells were sorted one cell per well into 96-well microtitre plates containing 100 μl of 50:50 fresh and conditioned complete medium with 200 μg/ml G418 and 50 μM ZnSO₄. The fluorescence intensity of cells could also be determined using a fluorescent plate reader or fluorometer (fmax, Molecular Devices, Sunnyvale, Calif.). This fluorometer equipped with a quartz halogen lamp has a filter set of 485 nm and 538 nm sufficient to detect the excitation and emission spectra of GFP. SOFTmaxPRO software was used to perform analysis on microtitre plates.

### EXAMPLE 7

### ELISA

To measure hGH, the conditioned media from cells grown in 96 microtitre plates for 10 days was centrifuged and hGH was quantified using ELISA (Roche, Mannheim, Germany). CAT protein levels were determined using ELISA (oche, Mannheim, Germany) as previously described (12)

### Results

Expression of MTGFP

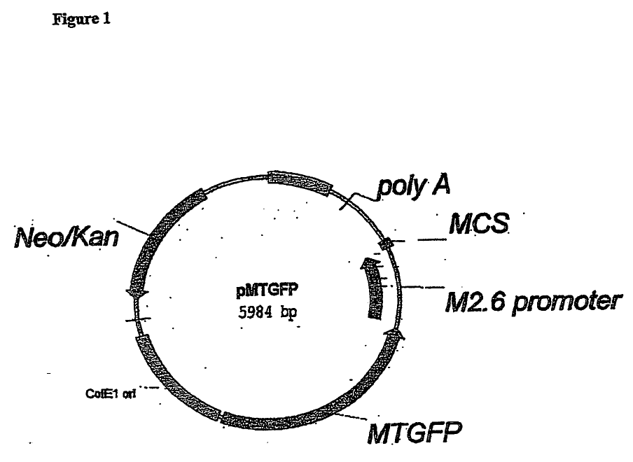

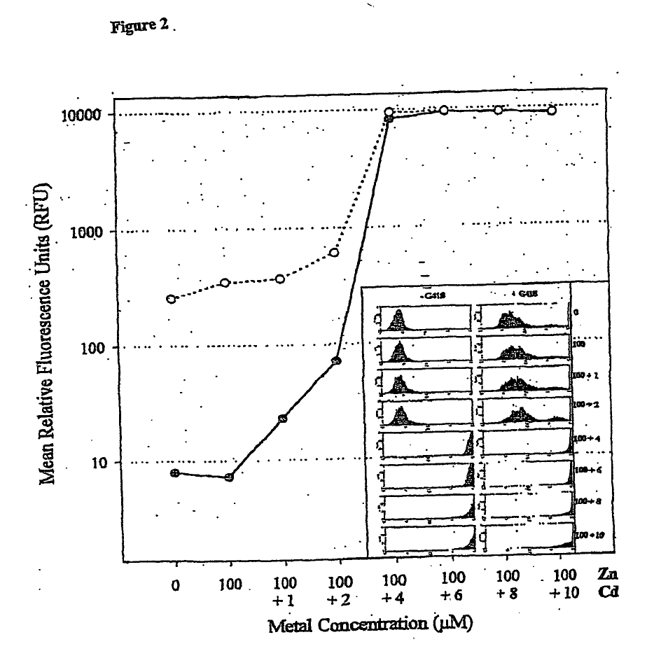

The expression of the fusion protein MTGFP was examined in CHO cells transfected with the plasmid vector pMTGFP (FIG. 1). The expression vector pMTGFP was constructed from the vector pNK (12) and differs from pNK in that the DNA encoding metallothionein IIA was replaced by that of the fusion gene retaining the entire promoter region of the metallothionein IIA gene. The modified metallothionein M2.6 promoter drives expression of a target gene cloned into the multiple cloning site (MCS). Pools of cells surviving selection in either the neomycin analogue, G418 or in metal were analysed using flow cytometry. Fluorescence was measured from pools of cells surviving various concentrations of Cd⁺⁺ (1-10 μM) in the presence or absence of G418. FIG. 2 shows the mean relative fluorescence of selected pools as a function of increasing metal selection. Each pool represents 10,000 single viable cells following 8 days selection in media containing the indicated metal concentrations in the presence or absence of G418. At low (100 μM Zn⁺⁺) or no metal selection there exists a 2- fold difference in mean fluorescence depending on whether G418 was used for selection. It appears that the addition of G418 alone is more effective at enriching a selected population than at low concentrations of metal (100 μM Zn⁺⁺+1-2 μM Cd⁺⁺). However, when cells are selected in metal containing 100 μM Zn⁺⁺+4 μM Cd⁺⁺, mean fluorescence increases 3 orders of magnitude above background levels. Background fluorescence seen in flow cytometry profiles of untransfected CHO cells appears as a peak corresponding to an average fluorescence of 7 relative fluorescent units (RFU) FIG. 2 inset.

Following 8 days under selection at this concentration of metal, all cells in the population are fluorescing at 10⁴ RFUs regardless of whether G418 was present in the media. There is in fact no peak corresponding to background fluorescence. Results were similar for cells selected at higher concentrations of metal. However, selection in 8 and 10 μM Cd⁺⁺ resulted in considerable cell death and therefore necessitated recovery of surviving cells in medium containing 100 μM Zn⁺⁺ and 2 μM Cd⁺⁺ in order to obtain enough cells to analyse in the flow cytometer.

It was sometimes necessary to attenuate light without discriminating wavelengths. This was done using a neutral density filter ND 1.3 (Company), which absorbed light over the entire visible spectrum. By using a filter that limits light reaching the photo multiplier tube, it was possible to reduce the signal considerably, thus obtaining values for those cells exhibiting fluorescence beyond 10000 RFUs (data not shown).

These results indicate that MTGFP can be used as an effective dominant selectable marker and that cells transfected with the expression vector encoding the MTGFP fusion protein can be efficiently and rapidly selected. Selection in 4 μM Cd⁺⁺ resulted in a pool of cells having 100 times more fluorescence than if selection was in G418 alone.

The MTGFP Fusion Can be Used as a Dominant and Visual Selectable Marker for Co-Expressed Genes

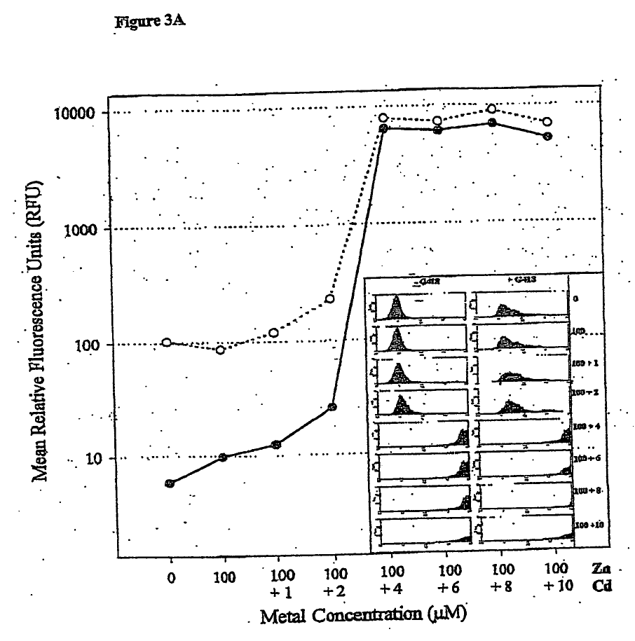

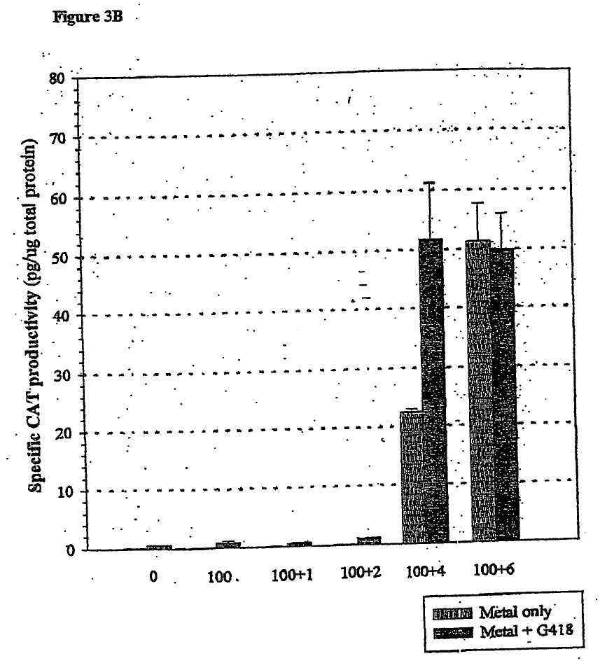

To evaluate the use of MTGFP fusion protein as a selectable marker for enriching high producing clones the reporter gene, chloramphenicol acetyl transferase (CAT) was co-expressed with MTGFP under the control of the modified M2.6 metallothione promoter (17). The CAT gene was cloned in the multiple cloning site of the expression vector pMTGFP and named pMTGFP/CAT. CHO cells were transfected with pMTGFP/CAT and selected in medium containing various concentrations of metal with or without G418 as described in the previous section. A graph representing the flow cytometry profiles of surviving transfected pools following 8 days of selection is shown in FIG. 3a. Again, as seen in the previous section, a dramatic enrichment of fluorescent pools was accomplished in concentrations of 4 μM Cd⁺⁺+100 μM Zn or higher. CAT expression levels from each pool under different selective pressure was analysed using a CAT ELISA (Roche, Australia) and the results are shown in FIG. 3b. CAT protein levels rose 20 times higher in those cells selected in media containing 4 μM Cd⁺⁺+100 μM Zn reflecting the fluorescent measurements from the flow cytometer and 50 times higher when G418 was included in the media These results indicate that cells transfected with the gene encoding MTGFP fusion protein can be efficiently selected in metal for the enrichment of pools with high fluorescence and high productivity. Due to the cytotoxicity of cadmium at high concentrations, selection was carried out in 4 μM Cd⁺⁺ and 100 μM Zn⁺⁺ for all subsequent experiments.

Expression of MTGFP Can be Used to Visually Monitor Gene Amplification in Increasing Metal

In addition to providing a marker for cadmium resistance, the amplification of the recombinant gene expression can be continued with stepwise increases of cadmium, which leads to increased expression beyond the initial levels. We have previously demonstrated the increase in CAT production in progressively higher levels of cadmium resistant pools of CHO cells tansfected with the expression vector pNK which encodes the metallothionein gene (12). Pools of transfected cells resistant to 120 μM cadmium resulted in over 500-fold increase in CAT gene expression over initial levels. However, it has been shown that stepwise selection reaching such high levels of cadmium gives rise to resistant cell populations resulting in a decline in productivity (data not shown) and (11). A similar pattern of expression is also seen when the DHFR system is used where an increase in MTX concentration often results in the loss of specific productivity Wurm (1990), Kaufman et al (1985). Studies using the DHFR amplification system suggest that there is an upper limit for MTX amplification and differs for each recombinant CHO cell line. The presence of a green fluorescent metallothionein gene enables the visual screening of highly amplified and cadmium resistant clones using the flow cytometer and/or the fluorometer. The appearance of cells that lose fluorescence was detected at high concentrations of cadmium. Loss of fluorescence clones in a pool of highly amplified cadmium-resistant transfected CHO cells can be easily sorted from the rest of the fluorescent population before their possible growth advantage results in the dilution of a promising high-producing pool.

Expression of hGH Correlates with GFP Fluorescence

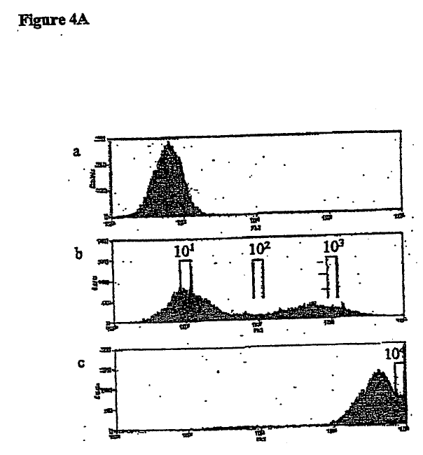

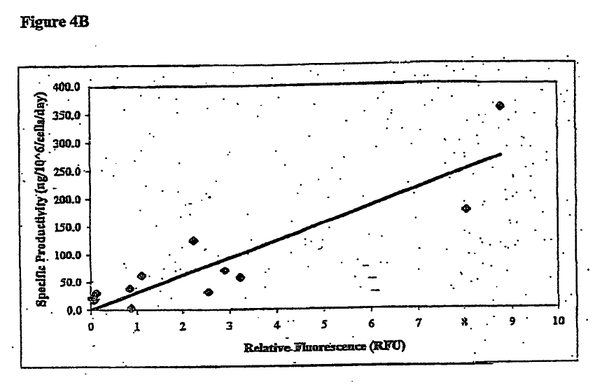

CHO cells were tansfected with pMTGFP/hGH and subjected to selection in either G418 (400 μg/ml) or metal (medium containing 100 μM Zn⁺⁺ and 4 μM Cd⁺⁺). After five days in selection media, the cells were analysed using the flow cytometer and gated according to their relative fluorescence intensities of 10¹, 10², 10³ and 10⁴ as shown in FIG. 4A. The flow cytometry profiles reveal that metal selection results in an average relative fluorescence of 4500, whereas a G418-resistant pool is comprised of cells with varying fluorescence intensities averaging 345 RFUs. This again indicates that metal selection is faster and more efficient at selecting high fluorescing cells than G418 alone. Cells were sterile sorted into 96-well microtitre tissue culture plates, one cell per well from within the gated regions indicated in the profiles in FIG. 4A. Twelve clones of various fluorescence intensities were isolated after FACS sorting and grown to confluence in microtitre plates. The GFP fluorescence of each clone was measured using a fluorometer and plotted against the respective hGH levels in the conditioned media as measured by an ELISA (Roche, Australia). FIG. 4B shows hGH productivity of each clone as a function of GFP fluorescence. It was observed that the measured fluorescence of individual clones that had been sorted from within one gated region by flow cytometry could be differentiated according to their fluorescence levels. More importantly, mean fluorescence of a clonal population of cells measured by flow cytometry corresponded well with fluorescence as measured by the fluorometer.

A trend emerging from multiple replicates of each clone indicates that the fluorescence of GFP as detected by the fluorometer showed a linear relationship with respect to specific hGH productivity of each clone. That is to say that clones that were sorted with high fluorescence intensities by flow cytometry analysis were relatively high producers of hGH and vice versa. Regardless of FACS sorting, if clones exhibited high fluorescence detected by the fluorometer, they were in turn relatively high producers of hGH. Subsequent rounds of flow cytometry analysis were done to confirm that each clone displayed a well-defined fluorescence peak at the intensity that it was originally sorted. The flow cytometry profiles of these clones remained the same after two weeks in culture and did not change when they were frozen and recultured.

A Relative Measure of Fluorescence: Flow Cytometer vs. Fluorometer

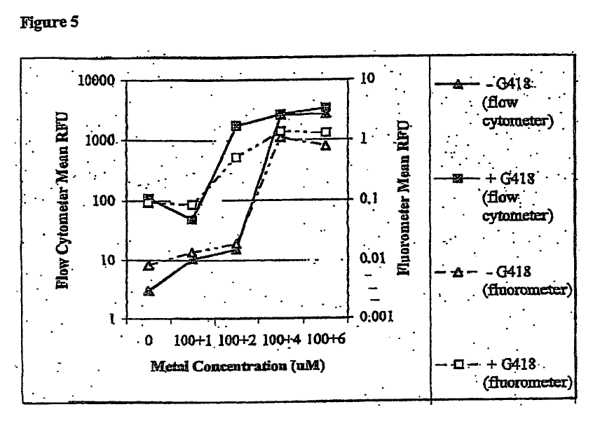

An experiment was done to demonstrate the potential for using the fluorometer to measure differences in fluorescence of clonal isolates. Sterile FACS sorting into microtitre plates is a rapid and convenient method for obtaining clones from a transfected population. After allowing individual clones to expand, a second measurement of fluorescence was done simply by scanning the microtitre plate in a fluorometer. This second round of selection was done to confirm the fluorescent nature of individual clones and allowed a further measure of comparison among clones in culture. For the application of a rapid screening process, it was important to establish that the fluorescence of clones determined by the flow cytometer was reproducible in the fluorometer. FIG. 5 is a plot of average fluorescence values obtained from clones examined in the flow cytometer compared to that of the fluorometer following isolation in different selection media These results demonstrate that although units of fluorescence are different as measured using the flow cytometer compared to that of the fluorometer, average fluorescence correlates very well between the two.

High Throughput Selection Protocol for High Producing Clones

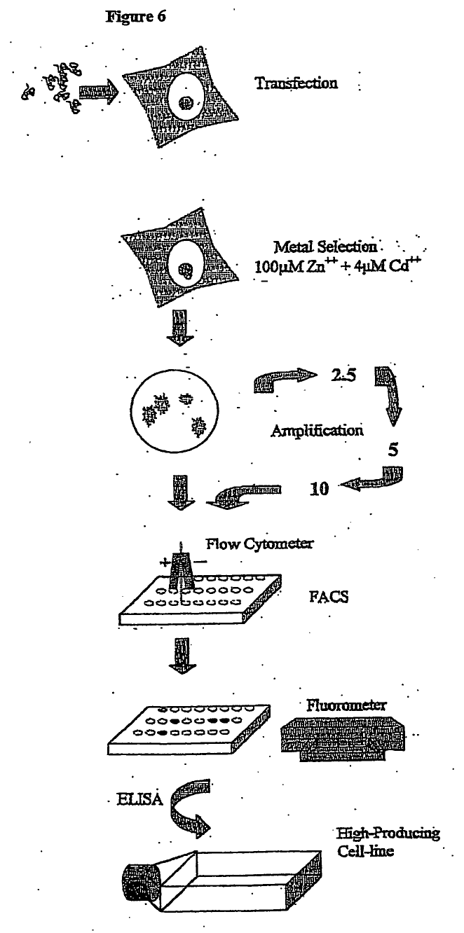

A very quick method can be employed for the selection of very high producing clones. A flow diagram depicting the process of rapid selection is shown in FIG. 6. This method is based on the correlation of GFP fluorescence and specific recombinant protein productivity. CHO cells are transfected with the plasmid vector pMTGFP expressing the desired gene. Following selection in metal (a recommended concentration would be 100 μM Zn⁺⁺ and 4 μM Cd⁺⁺ with or without G418) a pool of metal resistant cells is obtained which can be further amplified in increasing concentrations of Cd⁺⁺. Highly fluorescent cells are identified and sorted using FACS into one or several microtitre plates. Alternatively, clones can be obtained by limit dilution plating into microtitre wells. After 10 days in culture, cells in the microtitre plates are then scanned in the fluorometer. Since fluorescence resulting from the expression of MTGFP is a reliable indicator of productivity, the highest fluorescing clones can be picked for clonal expansion and further analysis. At this stage, conditioned media can be analysed using ELISA to determine recombinant protein expression. Alternatively, after selection/amplification in metal, limiting dilution into one or several microtitre plates can be performed, thus avoiding the requirement for expensive FACS equipment. Using the protocol as depicted in FIG. 6, it was possible to isolate cell lines with specific productivity >30 μg/10⁶ cells/day.

### Conclusion

A fusion MTGFP gene with which selection and amplification properties have been combined allows an efficient visual screening process of foreign gene positive clones with high levels of expression. The fusion protein is a dominant and visible marker for the selection and amplification of expression. Fluorescence correlates not only with amplification of the nucleic acid but also with productivity and therefore, high expressors can be identified according to their fluorescence levels. This work describes a high throughput screening method to identify high producing clones using a metallothionein-green fluorescent protein marker gene and flow cytometry. The method can be adapted for automation using robotic systems capable of selecting the highest producing clones among tens of thousands of transfected cells.

Although the invention has been described with reference to specific examples, it will be appreciated by those skilled in the art that the invention may be embodied in many other forms.

### REFERENCES

(1) Alt, F. W.; Kellems, J. R.; Bertino, J. R.; Schimke, R. T. Selective multiplication of dihydrofolate reductase genes in methotrexate-resistant variants of cultured murine cells. J Biol Chem 1978, 253, 1357-1370.
(2) Cockett, M. I.; Bebbington, C. R.; Yarranton, G. T. High level expression of tissue inhibitor of metalloproteinases in Chinese hamster ovary cells using glutamine synthetase gene amplification. Bio/Technology 1990, 8, 662-667.
(3) Urlaub, G.; Chasin, L. A. Isolation of Chinese hamster cell mutants deficient in dihydrofolate reductase activity. Proc Natl Acad Sci USA 1980, 77, 4216-4220.
(4) Brown, M. E. Process Development for the Production of Recombinant Antibodies Using the Glutanine Synthetase (GS) System. Cytotechnology 1992, 9, 231-236.
(5) Bebbington, C. R. High-level Expression of a Recombinant Antibody from Myeloma Cells using a Glutamine Synthetase Gene as an Amplifiable Selectable Marker. Bio/Technology 1992, 10, 169-175.
(6) Cockett, M. I.; Bebbington, C. R.; Yarranton, G. T. High level expression of tissue inhibitor of metalloproteinases in Chinese hamster ovary cells using glutamine synthetase gene amplification. Bio/Technology 1990, 8, 662-667.
(7) Gounari, F.; Banks, G. R; Khazaie, K.; Jeggo, P. A.; Holliday, R. Gene reactivation: a tool for the isolation of mammalian DNA methylation mutants. Genes Dev 1987,-1, 899-912.
(8) Harris, M. High frequency induction by 5-azacytidine of proline independence in CHO K1 cells. Somatic Cell Mol Genet 1984, 10, 615-624.
(9) Stallings, R. L.; Crawford, B. D.; Tobey, R. A.; Tesmer, J.; Hildebrand, C. E. 5-azacytidine-induced conversion to cadmium restance correlates with early S phase replication of inactive metallothionein genes in synchronized cells. Somatic Cell Mol Genet 1986, 12, 423-432.
(10) Beach, L. R.; Palmiter, R. D. Amplification of the metallothionein-l gene in cadmium-resistant mouse cells. Proc Natl Acad Sci USA 1981, 78, 2110-2114.
(11) Kushner, P. J.; Hort, B.; Shine, J.; Baxter, J. D.; Greene, G. L. Construction of cell lines that express high levels of the human estrogen receptor and are killed by esstrogens. Mol Endocrinol 1990, 4, 1465-1473.
(12) Bailey, C. J.; Baig, M.; Gray, P. P.; Sunstrom, N.-A. A rapid selection/amplification procedure for high-level expression of recombinant protein in a metal-amplifiable mammalian expression system. Biotechnol Techniques 1999, 13(9), 615-619.
(13) Chalfie, M.; Tu, Y.; Euskirchen, G.; Ward, W. W.; Prasher, D. C. Green fluorescent protein as a marker for gene expression. Science 1994, 263, 802-805.
(14) Mosser, D. D.; Caron, A. W.; Bourget, L.; Jolicoeur, P.; Massie, B. Use of a dicistronic expression cassette encoding the green fluorescent protein for the screening and selection of cells expressing inducible gene products. Biotechniques 1997,22, 150-154.
(15) Karin, M.; Richards, R. I. Human metallothionein genes: molecular cloning and sequence analysis of the mRNA Nature 1982, 299, 797-802.
(16) PCRPrimer A Laboratory Manual. Plainville, N.Y.: Cold Spring Harbor, 1995.
(17) McNeall, J.; Sanchez, A.; Gray, P. P.; Chesterman, C. N.; Sleigh, M. J. Hyperinducible gene expression from a metallothionein promoter containing metal-responsive elements. Gene 1989, 76, 81-88

1. A method of identifying from a plurality of cells, a cell in which the copy number of a nucleic acid of interest is altered compared to others wherein
(a) the cell in which the copy number of the nucleic acid of interest is altered comprises the nucleic acid of interest and an amplifiable nucleic acid linked to a reporter nucleic acid; and
(b) the copy number of the nucleic acid of interest is correlated with the copy number of the amplifiable nucleic acid and the reporter nucleic acid; and
wherein the method comprises the step of screening the plurality of cells for a cell having an alteration in copy number of the reporter nucleic acid and/or an alteration in expression of the product of the reporter nucleic acid compared to the other cells.

2. A method according to claim 1 wherein the nucleic acid of interest and the amplifiable nucleic acid linked to the reporter nucleic acid are on a single DNA molecule.

3. A method according to claim 1 wherein the nucleic acid of interest is 10,000 bp or less from the amplifiable nucleic acid.

4. A method according to claim 1 wherein the nucleic acid of interest is 1,000 bp or less from the amplifiable nucleic acid.

5. A method according to claim 1 wherein the alteration in expression of the product of the reporter nucleic acid correlates with the alteration in expression of the product of the nucleic acid of interest.

6. A method according to claim 1 wherein the alteration in copy number of the nucleic acid of interest is an amplification of the copy number.

7. A method according to claim 1 wherein the copy number of the amplifiable nucleic acid is regulated by the concentration of metal ion present in the growth medium of the cell.

8. A method according to claim 1 wherein the copy number of the amplifiable nucleic acid is amplified by increasing the metal ion concentration of the growth medium of the cell.

9. A method according to claim 1 wherein the copy number of the amplifiable nucleic acid is amplified by increasing the metal ion concentration of the growth medium of the cell from a low to a higher concentration both within a range of 1 μM to 100 μM.

10. A method according to claim 1 wherein the copy number of the amplifiable nucleic acid is amplified by increasing the metal ion concentration from 2.5 μM to 5.0 μM.

11. A method according to claim 7 wherein the metal ion is cadmium, zinc, copper, cobalt or nickel ion.

12. A method according to claim 11 wherein the metal ion is cadmium or zinc ion.

13. A method according to claim 1 wherein the amplifiable nucleic acid encodes a product that can act as a selectable marker.

14. A method according to claim 1 wherein the amplifiable nucleic acid encodes a metallothionein (MT), dihydrofolate reductase, glutamine synthetase gene, CAD, adenosine deaminase, adenylate deaminase, UMP synthetase, IMP 5′-dehydrogenase, zanthine-guanine phosphoribosyltransferase, mutant HGPRTase or mutant thymidine kinase, thymidylate synthetase, P-glycoprotein 170, ribonucleotide reductase, glutamine synthetase, asparagine synthetase, arginosuccinate synthetase, ornithine decarboxylase, HMG-CoA reductase, N-acetylglucosaminyl transferase, threonyl-tRNA synthetase, Na+, K+-ATPase.

15. A method according to claim 1 wherein the product of the reporter nucleic acid is detectable by flow cytometry, florescence plate reader, fluorometer, microscopy, the naked eye or phenotypic detection.

16. A method according to claim 15 wherein phenotypic detection is detection of the ability to grow in the presence of an inhibitor.

17. A method according to claim 16 wherein the inhibitor is neomycin, ampicillin, hygromycin, puromycin, bleomycin, zeocin or kanomycin.

18. A method according to claim 1 wherein the product of the reporter nucleic acid is detectable by flow cytometry using a florescence activated cell sorter (FACS).

19. A method according to claim 1 wherein the reporter nucleic acid encodes a green fluorescent protein (GFP), or a derivative thereof.

20. A method according to claim 19 wherein the derivative of the GFP is an enhanced green fluorescent protein (EGFP), a yellow fluorescent protein (YFP), an enhanced yellow fluorescent protein (EYFP), a blue fluorescent protein (BFP), an enhanced blue fluorescent protein (EBFP), a cyan fluorescent protein (CFP), an enhanced cyan fluorescent protein (ECFP) or a red fluorescent protein (dsRED).

21. A method according to claim 20 wherein the derivative of the GFP is the fluorescent protein EGFP.

22. A method according to claim 1 wherein the nucleic acid of interest and the amplifiable nucleic acid linked to the reporter nucleic acid are inserted into the cell by transformation or transfection of the cell with the nucleic acids.

23. A method according to claim 22 wherein the nucleic acids are on a single DNA construct.

24. A method according to claim 22 wherein the nucleic acids are on two or more constructs.

25. A method according to claim 23 wherein the single DNA construct is a plasmid.

26. A method according to claim 25 wherein the plasmid is a vector derived from the pNK plasmid

27. A method according to claim 1 wherein the reporter nucleic acid is located downstream of the amplifiable nucleic acid.

28. A method according to claim 27 wherein the amplifiable nucleic acid is linked to the reporter gene by in frame fusion of the two nucleic acids such that a fusion product is produced.

29. A method according to claim 28 wherein the fusion product is MTGFP.

30. A method according to claim 1 wherein the nucleic acids are linked by an internal ribosome entry site (IRES).

31. A method according to claim 30 wherein the IRES is an attenuated IRES.

32. A method according to claim 1 wherein the product of the nucleic acid of interest is part of a fusion product.

33. A method according to claim 32 wherein the fusion product includes a protease site or a tag to aid purification.

34. A method according to claim 33 wherein the tag is a 6-His tag or a glutathione-S-transferase tag or a peptide epitope.

35. A method according to claim 34 wherein the detectable epitope is a Flag or Hemophilus influenza epitope.

36. A method according to claim 1 wherein the cell is a mammalian cell.

37. A method according to claim 36 wherein the cell is a Chinese Hamster Ovary cell (CHO).

38. A method according to claim 37 wherein the cell is a CHOK1 cell.

39. A method according to claim 1 wherein the nucleic acid of interest is a nucleic acid encoding an antibody, a biopharmaceutical, an endonuclease, a methylase, an oxidoreductase, a transferase, a hydrolase, a lysase, an isomerase or a ligase, a storage polypeptide, a transport protein, an antigen or antigenic determinant, a protective or defence protein, a hormone, a structural protein, a protease or a synthetic polypeptide of interest or part thereof.

40. A method of identifying a cell expressing a polypeptide of interest comprising:
(a) expressing the polypeptide of interest in a cell in which expression of the polypeptide is correlated with expression of a reporter nucleic wherein the reporter nucleic acid is linked to an amplifiable nucleic acid;
(b) identifying the cell expressing the polypeptide of interest from among other cells by monitoring expression of the reporter nucleic acid.

41. A method of isolating a polypeptide of interest comprising:
(a) transforming or transfecting cells with a construct or constructs harbouring the nucleic acid encoding the polypeptide of interest such that expression of the polypeptide of interest is correlated with expression of an amplifiable nucleic acid and a reporter nucleic acid and wherein the amplifiable nucleic acid and the reporter nucleic acid are linked;
(b) increasing expression of the amplifiable nucleic acid;
(c) selecting a cell expressing the product of the reporter nucleic acid; and
(d) isolating the polypeptide of interest therefrom.

42. A method according to claim 1 wherein the amplifiable nucleic acid is the gene encoding MT.

43. A method according to claim 1 wherein the reporter nucleic acid is the gene encoding GFP or a derivative thereof.

44. A method according to claim 43 wherein the MT and GFP genes are fused in frame.

45. A method according to claim 1 wherein the method is used in robotic screening and/or protocols for high throughput selection of cells producing high levels of a product of interest

46. A cell comprising (a) a nucleic acid of interest, and (b) an amplifiable nucleic acid linked to a reporter nucleic acid, wherein the copy number of the nucleic acid of interest is correlated with the copy number of the amplifiable nucleic acid and the reporter nucleic acid.

47. A method of altering the copy number of a nucleic acid of interest in a cell according to claim 46, comprising exposing the cell to a factor that alters the copy number of the amplifiable nucleic acid.

48. A construct comprising a nucleic acid of interest and an amplifiable nucleic acid linked to a reporter nucleic acid such that when the construct is present in a cell, the copy number of the nucleic acid of interest is correlated with the copy number of the amplifiable nucleic acid and the reporter nucleic acid.

49. A cell identified by a method according to claim 1.

50. A polypeptide of interest isolated by a method according to claim 41.

51. A method according to claim 1 wherein the amplifiable nucleic acid encodes the human MT gene.

52. A method according to claim 51 wherein the human MT gene is the human metallothionein IIA gene.
